(19) United States
(12) Patent Application Publication
(10) Pub. No.: US 2002/0150632 A1
(43) Pub. Date: Oct. 17, 2002

(54) Psychonutracological method for craving reduction in humans

(76) Inventor: Nick Scott, Santa Barbara, CA (US)

Correspondence Address: Nick Scott, 201 Bath #2, Santa Barbara, CA, 93101, US

(21) Appl. No.: 10/095,216

(22) Filed: Mar. 12, 2002

Related U.S. Application Data
(60) Provisional application No. 60/268,007, filed on Apr. 12, 2001.

(51) Int. Cl. A61K 33/06; A61K 31/714; A61K 31/198; A61K 31/685; A61K 31/555; A61K 31/525

(52) U.S. Cl. 424/682; 514/52; 514/251; 514/276; 514/419; 514/561; 514/393; 514/78; 514/356; 514/184

(57) ABSTRACT

A method for reducing or eliminating cravings associated with habitual substance abuse by administering to a human a synergistic composition containing an effective amount of each of Glutamine, gamma aminobutyric acid, tyrosine, taurine, theanine, 5-hydroxtryptamine, phenylethylamine, phosphatidyl serine, phosphatidyl choline, inositol, thiamin, riboflavin, niacin, pyridoxin, folic acid, biotin, pantothenic acid, cyanocobalamin, chromium polynicotinate magnesium, and decosahexaenoic acid. The pharmaceutical composition can be administered after complete cessation of a given habit forming substance, or to nullify cravings and to reduce abuse of a given habit forming substance, without total and complete remittance prior to administration of the composition. Worthy of note, all constituents found in this composition may be derived by synthetic means, or from natural sources.

### FIELD FOR THE INVENTION

The present invention relates to a composition that is useful for drug craving reduction in humans and as a treatment for addiction or habitual use to a variety of substances. More specifically the invention is directed toward a composition containing 4 subgroups of nutrients, each grouping implicating it's distinctive role in craving reduction and overall recovery from physical addiction. Furthermore the invention is a composition containing a B-vitamin complex, neurologically active amino acids, and specific lipids that synergistically elicit reduced withdrawal symptoms, and normalize the neurochemical imbalance caused by chronic, habitual use of a physically detrimental substance.

### DESCRIPTION OF THE BACKGROUND

Substance abuse and addiction is an age-old dilemma that has plagued many societies. Many, many methods have been contrived to help people stop the cycle of abuse. These methods include everything from psychotherapy, to group congregations, to replacement drug treatment, and other varying nutritional practices. These methods have varying degrees of efficacy and no one method is right for every individual. Frequently, more than one method should be simultaneously administered. There is continuing research for the biological, environmental, and evolutional roots of substance abuse, and they cannot be separated into different entities, because of their high correlation factor.

Substance abuse is a broad and widely encompassing term, and within this patent document the term may be used to describe the habitual use of a variety of chemical compounds, some very common and fairly benign, and others highly dangerous, and relatively uncommon. The composition may be used to nullify cravings for such chemical substances consumed by humans such as refined sugar, caffeine, alcohol, nicotine, ephedrine, meth-amphetamines, opiates, and cocaine. It has been suggested that genetics play a role in substance abuse, and that certain individuals have a propensity towards chronic, frequent use where others are only prone to experimental or sporadic use. Propensity towards addiction can be considered a weak link in genetics, but it can also be attributed to environmental stimuli, such as societal images, peer pressure, behavioral stereotypes of different age groups, and ethnic cultural background.

This method for reduction of cravings associated with chronic habitual substance abuse is primarily concerned with the physical causes of addiction. It may be postulated that most case study individuals will have a combination of determining factors that have created their problem with chemical abuse. For the sake of clarity of scientific explanation, this document will be primarily concerned with the physiological mechanisms related to mental health or lack of.

One of the physiological mechanisms in which the composition works to reduce the dysphoria following the euphoria that many common drugs produce. Neurochemically, some individuals have below normal levels of serotonin, dopamine, and or other beta-endorphins that lends them to prolonged dysphorias and hence a greater craving for more of the substance of choice. This behavioral feature of some individuals is a distinctive characteristic of people with addictive personalities and effectively demonstrates why some individuals may be prone to abuse. In conclusion, a host of factors come into review when determining the origin of an addicts abuse, but one may conclude that physiologically, abnormalities exist regardless of immediate assumed cause, usually due to socioenvironmental factors.

Nutritionally, many practitioners have used a variety of supplements to promote normal serotonin or other neurotransmitter levels. Phosphatidyl Serine is well respected for its ability to boost serotonin. Taurine is known to boost the catecholamines noradrenaline and epinephrine for raised energy and awareness. Numerous studies have been published on the adrenergic, dopaminergic, and serotonergic effects that different nutrients play in the brain. The stress reducing effect has also been investigated. Tyrosine used alone, results in elevated adrenal hormones causing heightened irritability along with the positive energy boost. 5HTP used alone may cause drowsiness, associated with elevated serotonin levels. By example, these facts demonstrate why taking one or two neurologically active precursors can have both positive and unwanted effects. That is the motive and purpose of this method for craving reduction, the composition offers very complete, comprehensive brain nutrient supplementation and therefore comprehensive increases in neurotransmitter levels, naturally.

Furthermore, the invention at hand has a profile of neurologically active amino acids, B-vitamins, and lipids that supplies the human body with the essential building blocks necessary for both excitatory as well as inhibitory neurotransmitters.

This method of craving reduction is not only meant to reduce withdrawal symptoms, in attempt to reduce the immediate ill-effects associated with drug remittance, to combat relapse to the substance of choice, but also to supply the brain with nutrients that drug addicted individuals become deficient of due to poor diet and lifestyle choices. Thereby administering the invention at hand the end users will reap the benefits associated with taking phosphalipids, and neuroamines , neurotransmitter precursors and B vitamins and certain minerals. In our world today, it is not rational to assume that we can get all the nutrients our bodies need from the food supply. Hence the need for nutritional supplementation. Further, years of “artificial” stimulation of cognitive neural systems by drug users leads to the down regulation of neurochemistry, chemical balance. This may be corrected, if it is caught in an earlier stage, although the brain may be considered a highly resilient organ, and full recoveries from long term chronic abuse of cocaine, amphetamines, and opiates is not uncommon. Years of abuse can lead poor lifestyle behavior, including depleted dietary intake. The human brain can recuperate, if the nutrients required for neural tissue rebuilding and neurotransmitter synthesis are supplied in the concentrated, highly absorbable form, and of course time is given.

The pseudo hyperconcentrations of serotonin, dopamine, and beta-endorphins, chemicals that run the human brain's cognitive neural systems as a result of drug administration causes imbalance. Depression, anxiety, and a host of other mental ills may result. The composition at hand's primary function is to boost neurochemical levels with initial, immediate administration and effect, because cravings are the result of low levels of neurochemicals. This initial boost in neurotransmitter is the result of the B-vitamin complex subgroup of this formula. Secondarily, with intermediate term use depression will be alleviated allowing the individual to think rationally, bettering their chances of quitting their detrimental substance. With long-term use (2 plus months) the brain nutrients have been given time to start healing the brain on the neurological structural level. This means dendrital connections and communication will begin to improve. Integrity of myelin sheaths and axon s will become stronger. The ex-user will regain that sense of mental clarity they had forgotten existed, and they will no longer crave that artificial stimulation that they once did.

The said method for craving reduction in humans may be subclassiffied As such:

1. Lipids and substrates

2. B vitamins and select minerals

3. Amino acid precursors

### SUMMARY OF INVENTION

Accordingly, one object of the present invention is to provide immediate reduction of cravings for a variety of substances including, but not limited to; refined sugar, caffeine,weight loss stimulants, compulsive binge eating, alcohol, nicotine, opiates, barbiturates, amphetamines, and cocaine, Central Nervous System stimulants, and C.N.S. depressants. A further object of this synergistic composition is to help improve the rate of total remittance of a substance when it is completely stopped at once. Improving the rate of total remittance may also be described as reducing relapse potential. A further objective is to provide an immediate boost in positive mood enhancing naturally occurring neurotransmitters, and still further provide the building blocks needed for long term healing of neural tissue, thus improving cognitive processes and neurochemistry to aid in the healing process that will take months if not years fallowing drug abuse cessation. This composition speeds this process, and allows for the time after remittance to be less traumatic, and depressing, reducing chances for relapse.

These and other objectives will become apparent from the following specification which comprises numerous variations of the formula at hand. The present method comprises administering to a human a composition comprising effective amounts of each of (the ingredients) into a pharmaceutical craving reduction and neurally healing composition containing effective amounts of these compounds.

### BRIEF DESCRIPTION OF DRAWINGS

A more complete appreciation of the invention and many of the accompanying advantages thereof will be readily obtained from the drawings, wherein.

FIG. one is a detailed flow chart of the amino acids in this formulation, and their respective roles in monoamine and neurotransmitter synthesis. Although P-5-P is considered a cofactor by some, it is the most important cofactor found in neural tissue, and almost is as active as some neurotransmitters in brain tissue. FIG. one includes pyridoxal phosphate in the neurotransmitter column because it is so important to mental health. That is to say that exogenous sources of the precursor pyridoxine are needed for healthful levels of the active form of this b-vitamin; pyridoxal phosphate in the brain.

Many of the b-vitamins have similar carbohydrate metabolism function, but the flow chart does not exemplify all motives for including particular compounds in the formula, nor does it include all ingredients. For example; folic acid may not have a major effect on neurochemistry, and isn't included in the flow chart, but if it is not included in the formula, an imbalance will develop in regards to the b-vitamins and deficiency will occur. Like all the B-vitamins, one of folic acids main functions is carbohydrate metabolism, and glucose is the main supply of energy for the brain. If the brain does not have enough folic acid, glucose metabolic efficiency is hindered. Neurotransmitter levels are integrally linked to energy metabolism in the brain. In conclusion, cognitive efficiency is hindered when a deficiency is present in certain nutritional compounds, and the flow chart does not portray this important implication of all constituents in the invention at hand.

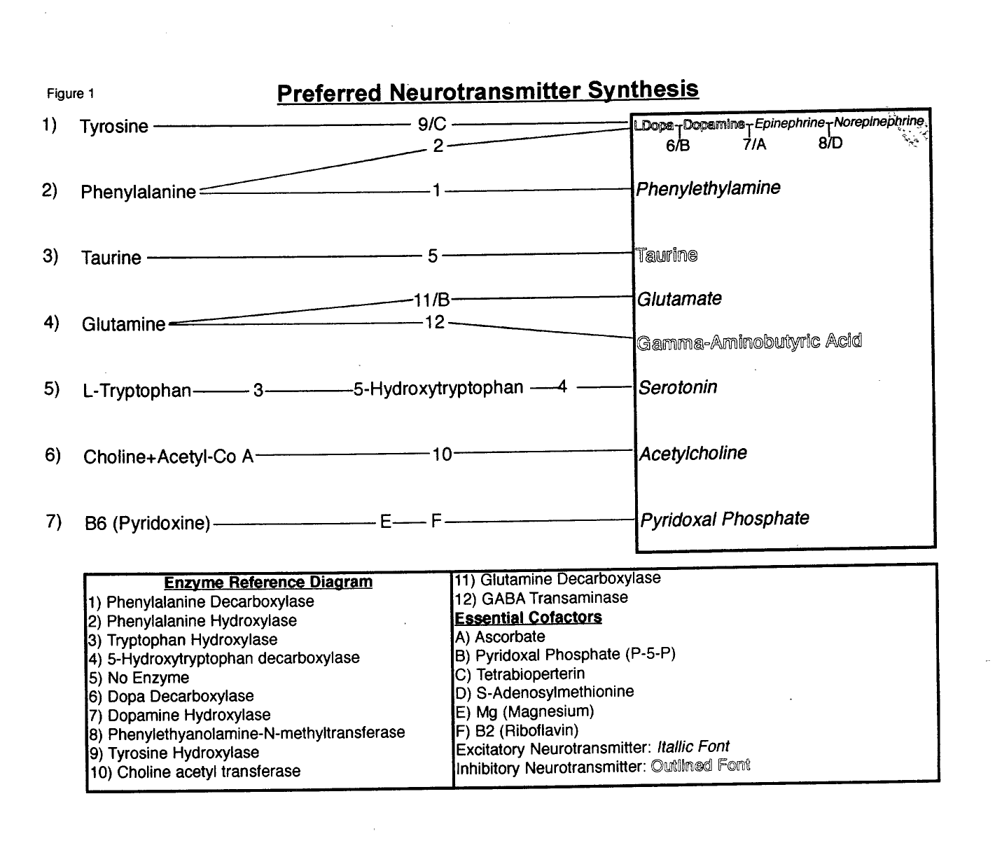

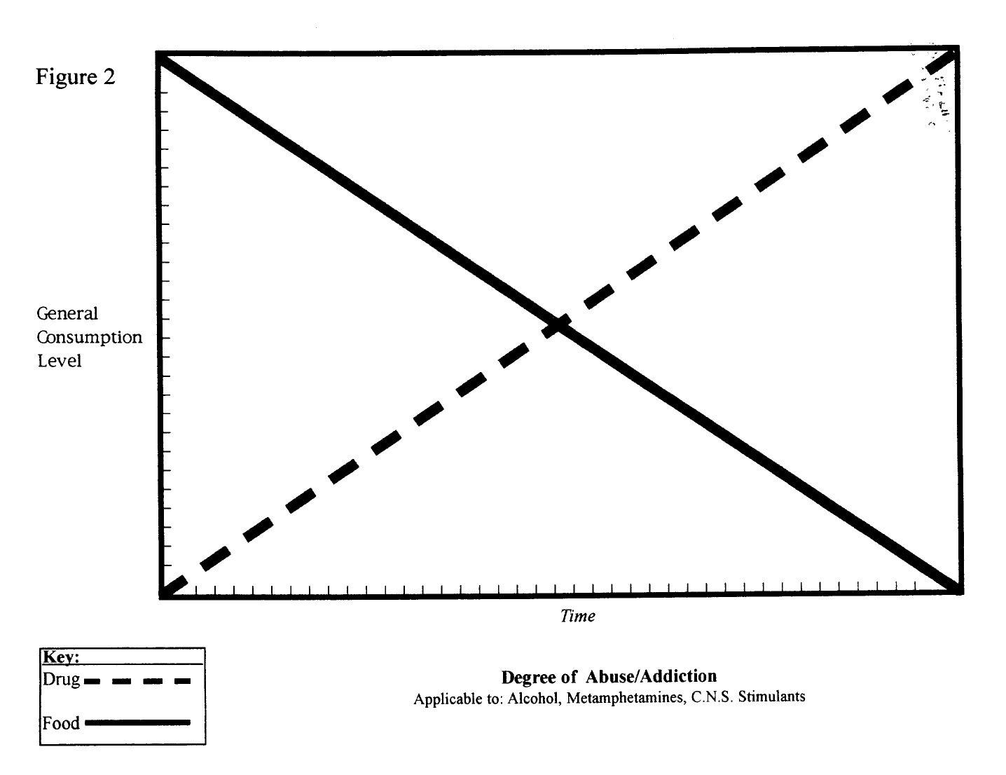

Tyrosine can be converted to either excitatory or inhibitory neurotransmitters. Pyridoxine is the key nutrient that enables production of the beneficial neuroamines dopamine and phenylethylamine. An important note is that B6 must first be converted into the active chemical pyridoxal phosphate (PLP) before it becomes a catalyst for amine production. As the flowchart (FIG. 1) exemplifies riboflavin (B2) and Magnesium (MG) are needed for this active conversion.

The essential amino acid glutamine has very important implications, because it passes through the blood brain barrier with ease and can converted into two other important neurotransmitters. It then can either be converted to Gamma aminobutyric acid or glutamate (glutamic acid). gamma aminobutyric acid is the most prevalent amino acid neurotransmitter that has an inhibitory effect on the brain. Glutamic acid is the most prevalent excitatory amino acid neurotransmitter. It has important functions in regards to potassium transport into the brain. Ideally, one wants a greater abundance of inhibitory modulating amines such as dopamine and phenyethlyamine to be in greater quantity than excitatory amities such as serotonin, epinephrine, and norepinephrine.

FIG. two represents the conceptual correlation between total caloric intake and total drug intake. As footnoted, this concept applies to one major group of drugs, central nervous system stimulants, and the carbohydrate; alcohol. One important implication of the concept portrayed by the graph is that over time one can assume that health deteriorates as drug consumption goes up, and food intake down. Thus, it can be extrapolated that essential nutrients found in the food supply become deficient and cognitive processes hindered. This is exactly what the high concentrations of brain nutrients found in the said invention works to correct.

Alcohol works on a different mechanism of appetite suppression than the central nervous system stimulant class of drugs. Alcohol is thought of by many to be an appetite stimulant, in the dining room setting, but chronic abuse of high quantities is much different scenario. In the respect that alcohol artificially stimulates, and suppresses mechanisms of the brain, it becomes the preferred metabolic compound to consume over food. Both are food and alcohol are calorie containing. Alcohol is a carbohydrate, and thus contains 4 calories per gram. Alcoholic beverages generally do not contain even moderate nutrient levels. Hard alcohol is the least nutrient dense, but contains 150 calories in an 1.5 ounce glass. In contrast, stout, thick beer has high b-vitamin levels, but alcohol content in the 3% range. Bottom line being that alcoholics substitute the “empty” calorie alcohol for nutrient dense food. This leads to chronic drinkers becoming deficient in many essential nutrients. Not to mention that alcohol has a leaching effect on nutrients from the body.

Amphetamines, cocaine derivatives, caffeine, and ephedrine have appetite suppressive side effects on individuals who take these substances. Amphetamines may be considered as a class to have stronger suppressing effects than cocaine. Methamphetamines have been prescribed as weight loss aids by doctors in earlier years. These types of drugs are obviously not calorie containing, and work via alternate mechanisms to that of alcohol to elicit appetite suppressive qualities.

### DETAILED DESCRIPTION OF PREFERRED EMBODIMENTS

The composition, of the present invention is useful for reducing cravings associated with substance abuse in humans that have developed dependency. A person who has a problem may be administered the composition under recommendation of a dietician or nutritionist, or an individual may recognize the need to quit smoking, or drinking multiple cups of coffee and self “prescribe” the formula. There is no concrete formula for discerning a problem user from a recreational user. In the case of more serious drug addiction, it is rather clear that there is a problem. This method for craving reduction, is broadly encompassing, and this is because many drugs work upon similar neurotransmitters and pathways in the human brain. The composition is highly effective in positively benefiting many neuroamines, and neurotransmitters. Not all neurotransmitters are effected by all drugs, although different classes of drugs work on their similar pathways in the brain. This formula is meant to positively effect overall brain chemistry in order to give the subject their best chance at recovery. Again it should be noted that the method for reducing cravings associated with chemical dietary intake does not artificially stimulate neurotransmitter release or production and carries no ill side effects because they are naturally occurring substances found in the readily available food supply. Of course, some may experience upset stomach or nausea associated with high concentrations of neurologically active compounds. The nutraceutical preparation is a highly concentrated source of neurologically active B-vitamins, neurologically active amino acids, and the building blocks for related elements found in neural tissue.

Hypoglycemia and hyperglycemia both have their respective affect on mood and cravings. Many Obsessive Compulsive binge eaters suffer from fluctuations in blood sugar as do 90% of alcoholics. When a subject with drug addiction or an eating disorder is in a hypoglycemic state they are in the danger zone for substance abuse. Higher peripheral cortex processes hindered, and poor judgement may be exercised. During recovery, it is important to regulate blood sugar levels to avoid relapse. To reiterate, cravings and hypoglycemia are synonymous terms in the world of substance abuse. Hypoglycemia lends itself to a low energy environment where cells do not have much glucose to fuel the citric acid cycle within cell mitochondria. This stunts cell function. In the case of soma cells, stunting function, elicits reduced output of cellular substances like neurotransmitters such as serotonin and adrenal hormones. Low serotonin and epinephrine levels translates to behavioral characteristics such as anxiety, lack of concentration, low energy, cravings for preferred substance, reduced motivation, and depression. One mineral in particular, chromium, has been found to increase sensitivity of cell receptorsites to insulin. Chromium has been shown to improve metabolism and regulate blood sugar vacillations. The invention at hand contains the most potent form of chromium, which is chromium polynicotinate. Regulating a recovering user's or binge eater's blood sugar has important implications. Glutamine found in the present invention is also helpful in fighting hypoglycemic effects on the brain because it is the only other fuel source the brain has. In the absence of proper levels of glucose, glutamine is utilized as fuel for somas and other neural bodies, therefore having important implications in regulating energy levels and good judgment when an individual has started a drug cessation program. It should also be mentioned that proper dietary practices in conjunction with the composition at hand improves success rate of permanent remittance, and reduces the chance for relapse. It is recommended that directly following administration of the nutraceutical composition one should eat a modest size meal low in sugar (low glycemic), and high in plant protein.

The first few years last a lifetime. A child's diet has life long ramifications on food preferences and behavior in regards to drug use that carry on into adulthood. There is a high correlation factor between addiction to drugs later in life, and high consumption of high glycemic carbs (predominately refined sugar) during youth. The brain of a child is much more sensitive to the neurologically active compounds that it is exposed to. It is common knowledge that the younger an individual is when they start using a psychoactive drug, whether it be nicotine or a more serious illegal drug, the higher their chances are for developing a habitual chronic use problem. A diet high in sugar for children effects their serotonin and overall brain chemistry much more substantially than adults. This is obvious by observation of a child who has consumed a highly concentrated source of sugar, like a soda. As we grow older, an adult individual typically notices heightened energy followed by lethargy when they consume high glycemic carbohydrates, but by observation one notices that the behavioral changes are not as radical. Due to conditioning, the child becomes accustomed to the sharp oscillations in blood sugar, and the highs and lows in mood associated with it. This mental environment carries onto into adulthood as the individual seeks drugs that elicit the same sharp mental response they once attained from sugar and caffeine laden candies and sodas.

High glycemic sugars elicit a serotonergic response, an increase in the stress hormones epinephrine and nor-epinephrine, along with the stress compounds glucocorticoids. During administration of the invention at hand, consumers will be informed of the importance of their overall diet.

The formulas efficacy is found in its comprehensive nutritional constituent profile, which allows for the rebalancing of neurochemistry, and improved structural integrity of neural bodies. It is necessary to supply the brain with nutrients that improve synthesis of both excitatory and inhibitory neurotransmitters, both catecholamines and indolamines. Because this formula requires natural conversion to preferred neurotransmitters, it will not create an “artificial” chemistry in human subjects as a counteracting or replacement drug therapy for addiction would. Myelinization of axons is very important to chemoelectrical nerve impulse transmission. The phosphalipids in this formula supply essential nutrients mandatory for humans to synthesize neuronal bodies, and also to maintain soma membrane integrity and plasticity. By supplying nutrients that support normal membrane texture and consistency normal absorption and communication efficiency is maintained.

More specifically, pyridoxine and magnesium are of paramount importance to the conversion of dietary amino acids to preferred amines. Magenesium and vitamin B deficiencies cause reductions in the production of dopamine. Magnesium deficiencies reduce levels of serotonin and norepinephrine. Pyridoxine is integral to the absorption of magnesium by neuron cells, and so it can be said that magnesium and B6 synergistically aid in production of catecolamines and serotonin.

Furthermore, it is important to note that the synergistic formulation is highly efficacious. Craving reduction is accomplished even in individuals who do not desire to stop consuming a given substance, weather it be a concentrated source of refined sugar or for a cigarette. In the case of a smoker, who does not desire to quit, administration of the composition would make smoking less desirable and quantity of cigarettes consumed would be decreased.

The origin of cravings is relatively uncharted, although generally a deficiency is present in one or more of the active neurotransmitters that elicit elative emotions. Hence negative emotions in the form of anxiety, frustration, anger, aggressiveness, depression, sadness, lack of energy are present until the deficiency is corrected artificially by the substance of choice. Of course this cycle is an endless “rollercoaster” of ups and downs in emotions and neurochemistry, commonly referred to by ex-users and psychotherapists alike. It is reasonable to assume that the composition at hand be implicated in treatment for addiction before more radical drug replacement methods fallow. Although replacement drug therapy has mixed reviews, and does not address the very common nutrient deficiencies that lend to addictive behavior as the result of compromised neural function, nerve transmission, and neurotransmitter synthesis.

Administration of the nutraceutical composition is best accomplished in a liquid form orally, Although the invention will also be administered in capsule form, orally. The preferred method is by effervescent liquid suspension for the most quickly bioavailable absorption and utilization by neural tissue for metabolism. Amino acid precursors and Phosphalipids preferentially will be administered by capsule.

What is claimed as new and desired to be secured by Letters Patent of the United States is:

1. A method for reducing the cravings associated with habitual, chronic chemical substance abuse or addiction in humans comprising the step of administering a therapeutically effective amount of Glutamine, gamma aminobutyric acid, tyrosine, taurine, theanine, 5-hydroxtryptamine, phenylethylamine, phosphatidyl serine, phosphatidyl choline, inositol, thiamin, riboflavin, niacin, pyridoxin, folic acid, biotin, pantothenic acid, cyanocobalamin, chromium polynicotinate and magnesium and decosahexaenoic acid.

2. A method as set forth in claim 1, wherein said chemical substance is alcohol.

3. A method as set forth in claim 1, wherein the chemical substance is nicotine.

4. A method set forth in claim 1, wherein the chemical substance is refined sugar.

5. A method as set forth in claim 1, wherein the chemical substance is an opiod or opiod derivative.

6. A method as set forth in claim 1, wherein the chemical substance is an amphetamine.

7. A method as set forth in claim 1, wherein the chemical substance is caffeine.

8. A method as set forth in claim 1, wherein the chemical substance is ephedrine.

9. A method set forth in claim 1, wherein the chemical substance is cocaine.

10. A method as set forth in claim 8, wherein the chemical substance ephedrine is naturally occurring in mau waung.

11. A method as set forth in claim 7, wherein the chemical substance caffeine is naturally occurring in the coffee bean or guarana.

12. A method set forth in claim 9, wherein derivative of the coke plant is abused by humans.

13. A method set forth in claim 1, wherein the proprietary synergistic formulation is administered orally in liquid form.

14. A method set forth in claim 1, wherein the proprietary synergistic formulation is administered orally in capsule or pill form.

15. A method for correcting nutrient deficiencies whereby orthomolecular nutrient supplementation improves structural integrity of neural tissues, thereby improving soma communication, and improving neurotransmitter communication.

16. A method for improving cognitive efficiency and mood, whereby both neurochemistry, and neural matter are improved via othomolecular nutritional psychology.

17. A method set forth in claim 16, wherein orthomolecular nutritional phychological practices implicated to formulate the said invention reduces cravings and relapse potential by correcting dysphoria, and raising catecolamines.

18. A method set forth in claim 16, wherein orthomolecular nutritional psychological practices implicated to formulate the said invention reduces cravings and relapse potential by correcting dysphoria, by raising the indolamine, serotonin.

19. A method set forth in claim 16, wherein orthomolecular nutritional psychological practices implicated to formulate the said invention reduces cravings and relapse potential by correcting amphetamine pathway agonistic neuromodulator levels, by exogenous supplementation of the endogenous, naturally occurring amino acids; phenylalanine and phenylethylamine.
